(19) United States
(12) Patent Application Publication
(10) Pub. No.: US 2021/0353796 A1
(43) Pub. Date: Nov. 18, 2021

(54) AUTOMATIC UVC SANITIZER FOR COMMON TOUCH POINTS

(71) Applicant: Gordon Pratt, Boise, ID (US)

(72) Inventors: Gordon Pratt, Boise, ID (US); Chandrappa Nataraj Bangalore, Bangalore (IN)

(21) Appl. No.: 17/317,409

(22) Filed: May 11, 2021

Related U.S. Application Data
(60) Provisional application No. 63/025,666, filed on May 15, 2020.

(51) Int. Cl. A61L 2/10; A61L 2/24; A61L 2/26

(52) U.S. Cl. CPC A61L 2/10; A61L 2/24; A61L 2202/14; A61L 2202/11; A61L 2/26

(57) ABSTRACT

A low-cost, low-power, motion-activated LED UVC light with intrinsic safety features to automatically sanitize a common touch point after it has been contacted by a human finger or hand.

### CROSS-REFERENCE TO RELATED APPLICATIONS

This application, under 35 U.S.C. § 119, claims the benefit of U.S. Provisional Patent Application Ser. No. 63/025,666 filed on May 15, 2020, and entitled “Automatic UVC Sanitizer For Common Touch Points,” the contents of which are hereby incorporated by reference herein.

### FIELD OF THE DISCLOSURE

The present disclosure relates to the technical field of electronic devices. More particularly, the present disclosure relates to the technical field of public health and sanitizing. More particularly, the present disclosure relates to the technical field of sanitizing objects touched frequently by humans.

### BACKGROUND

UV (ultraviolet) light, categorized as UVA, UVB, and JVC depending on wavelength, has been used to kill germs, bacteria, and viruses since the late 1800s. UV is a safe and effective means of sanitization. UVC (200-280 nm) is particularly effective.

Commonly touched items such as door handles, light switches, elevator buttons, faucet handles, and ATM keypads are transmission focal points for bacteria and viruses. Some existing solutions to attempt to sanitize such commonly touched items include devices that use UV bulbs arranged in parallel on each side of a door handle, devices that use accelerometers to detect when a door has moved in order to trigger the UV light, and devices that surround escalator railings to provide disinfection. Furthermore, there are whole-room and handheld wand UV light sanitizers.

However, these devices are typically not small enough or configured suitably to accommodate a wide variety of common touch points. Additionally, UV light bulbs are typically large and inefficient and generate significant heat, making them suboptimal for a compact, low-cost, low-powered application. Furthermore, prolonged exposure to UV light is harmful to humans. Other drawbacks, inefficiencies, and issues also exist with current systems and methods.

### SUMMARY

Accordingly, disclosed embodiments address the above-noted, and other, drawbacks, inefficiencies, and issues that exist with current systems and methods. Disclosed embodiments use low-cost, low-power LED UVC light, and a motion detector to automatically sanitize a common touch point after it has been touched. Also disclosed are methods that are employed to minimize human exposure to the UVC light. As used herein, “automatic,” “automatically,” and the like mean without the need for further human intervention to operate.

In one embodiment, the automatic sanitizer containing the UVC light source is attached adjacent to a set of elevator buttons. A UVC reflector may be placed on the opposite side of the buttons so that the buttons are between the UVC light source and the reflector so as to provide sufficient coverage for sanitation. For large arrays of elevators buttons, a plurality of the devices can be used to ensure sufficient coverage.

In another embodiment, the automatic sanitizer is placed adjacent to a door handle on an extender which enables the UVC light to illuminate the handle with an angle of incidence sufficient to provide sufficient coverage.

In yet another embodiment, the automatic sanitizer can be embedded within or attached to an AFM or gas pump so as to effectively sanitize the keypad or touchscreen between uses.

Disclosed embodiments also include an automatic sanitizer for use near a common human touch point, the automatic sanitizer including a power source to provide power to operate the automatic sanitizer, a motion sensor to detect the presence of a human appendage within a field of detection of the motion sensor and to generate a corresponding motion signal, and a microcontroller circuit that operates at least one UVC LED to illuminate after a first predetermined amount of time from receipt of the corresponding motion signal and to cease illumination of the UVC LED after a second predetermined amount of time or if presence of a human appendage is detected.

Further disclosed embodiments include an enclosure to house the power source and the microcontroller circuit, at least a portion of the motion sensor, and at least a portion of the UVC LED. Still further disclosed embodiments include an extender mount attached to a base of the enclosure and configured to provide an angle of incidence to illumination from the UVC LED onto the common human touch point near the automatic sanitizer.

In some embodiments the power source may comprise a battery. Further embodiments may include a low battery indicator to provide an indication when the battery power will be inadequate to operate. In some embodiments the low battery indicator may comprise a tight.

Some disclosed embodiments may include a safety mechanism to shield human eyes from direct exposure to the illumination from the UVC LED. Embodiments of the safety mechanism may comprise an appropriately shaped lip on the enclosure.

Some embodiments may include a reflector positionable near the common human touch point and configured to reflect illumination from the UVC LED onto the common human touch point.

Also disclosed is an automatic sanitizer system for use near a common human touch point, the automatic sanitizer system including a power source to provide power to operate an automatic sanitizer device, a motion sensor on the automatic sanitizer device to detect the presence of a human appendage within a field of detection of the motion sensor and to generate a corresponding motion signal, a microcontroller circuit in the automatic sanitizer device that operates at least one UVC LED to illuminate after a first predetermined amount of time from receipt of the corresponding motion signal and to cease illumination of the UVC LED after a second predetermined amount of time or if presence of a human appendage is detected, and a reflector positionable near the common human touch point across from the automatic sanitizer device and configured to reflect illumination from the at least one UVC LED onto the common human touch point.

Further disclosed embodiments of the system may also include an extender mount attached to a base of the automatic sanitizer device and configured to provide an angle of incidence to illumination from the UVC LED onto the common human touch point near the automatic sanitizer.

In some embodiments the power source may comprise a batter. In further embodiments, the system may include a low battery indicator to provide an indication when the battery power will be inadequate to operate. In still further embodiments, the low battery indicator may comprise a light.

Further disclosed embodiments of the system may include a safety mechanism to shield human eyes from direct exposure to the illumination from the UVC LED. In some embodiments, the safety mechanism may comprise an appropriately shaped lip on an enclosure of the automatic sanitizer device. Other embodiments also exist.

### BRIEF DESCRIPTION OF THE DRAWINGS

FIG. 1 is a perspective view of the automatic sanitizer arranged about a commonly touched object.

FIG. 2 is a transparent, perspective view of a portion of the automatic sanitizer showing features within.

FIG. 3 is a perspective view of an optional reflector.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the spirit and scope of the invention as defined by the appended claims.

### DETAILED DESCRIPTION

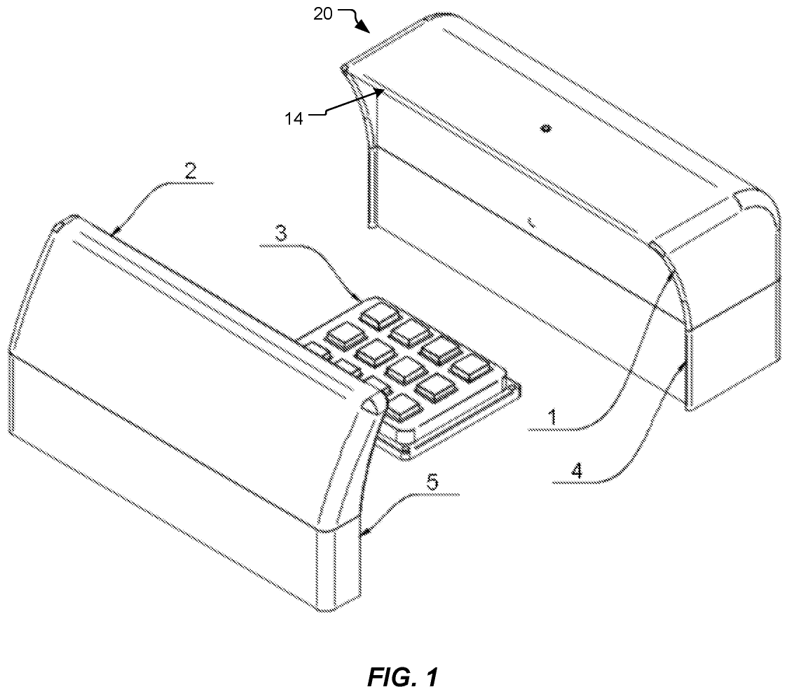

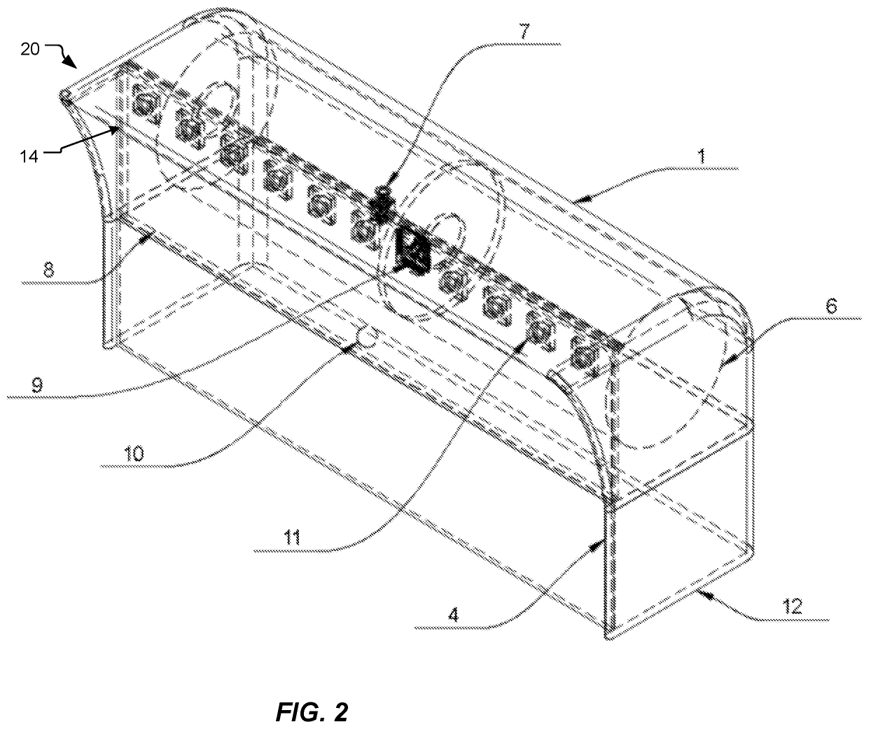

With reference to FIGS. 1 and 2 an automatic sanitizer 20 may be arranged about a commonly touched object 3 (in FIG. 1 shown as a keypad). Representative embodiments of the automatic sanitizer 20 may include an enclosure 1. In some embodiments enclosure 1 may comprise plastic or other lightweight, but durable, material. In other embodiments, the device may be embedded into another device or structure, such as an ATM machine, gas pump, or the like, without employing a dedicated enclosure 1.

As shown in FIG. 2, enclosure 1 houses a power source 6 (e.g., batteries) and a circuitry mount 8 (e.g., a printed circuit board, or the like). The circuitry mount 8 is populated with a motion sensor 10, microcontroller 9, one or more UVC LEDs 11, and a low battery indicator 7 (e.g., a red LED, or other alert indicator). Exemplary motion sensors 10 include passive infrared (PER) and microwave (MW) technology, or the like. Exemplary microcontrollers 9 include [examples?], or the like. Exemplary UVC LEDs 11 include [examples?], or the like.

The UVC LEDs 11 are arranged so as to concentrate the UVC light on the common touch point 3 which may be, but is not limited to, elevator buttons, keypads, light switches, door handles, faucets, toilet-flush handles, touch screen kiosks, vending machines, or the like. In some embodiments, an extender mount 4 may, be attached to the base of the enclosure 1 to provide a better angle of incidence to accommodate touch points 3 that extend above the touch point 3 mounting surface such as, but not limited to, a door handle. Embodiments of the extender mount 4 may also comprise plastic or other lightweight durable material. Depending upon the configuration, the base of either the enclosure 1 or the extender mount 4 is affixed securely to the surface adjacent to the touch point 3 to be sanitized. Affixing may be accomplished by fasteners (e.g., screws, bolts, or the like), by adhesives, by snap fit, or by any other suitable mounting method depending upon the surface and desired permanence or strength of affixing.

In operation, embodiments of the automatic sanitizer 20 work as follows. When motion of a finger, hand, or other human appendage is detected by the motion sensor 10, the microcontroller 9 waits for specified period (e.g., 1 second) then causes the UVC LEDs 11 to be illuminated for a specified period (e.g., 10 seconds) to sanitize the touch point 3. If unsafe proximity of a human is detected by the motion sensor 10 during the illumination period, the microcontroller 9 causes the UVC LEDs 11 to cease illumination so as not expose human skin to UVC light unnecessarily. The enclosure 1 may include an appropriately shaped lip 14 to shield human eyes from direct exposure to UV light. Alternatively, a movable shutter or other filter may flip or otherwise extend out during operation of the UVC LEDs 11 to shield human eyes, or some other safety mechanism may be employed.

For embodiments with batteries as the power source 6, the microcontroller 9 monitors battery charge and produces an alert on low battery indicator 7 to alert a low state of charge, such as a red LED, or the like, which is visible externally on the enclosure 1.

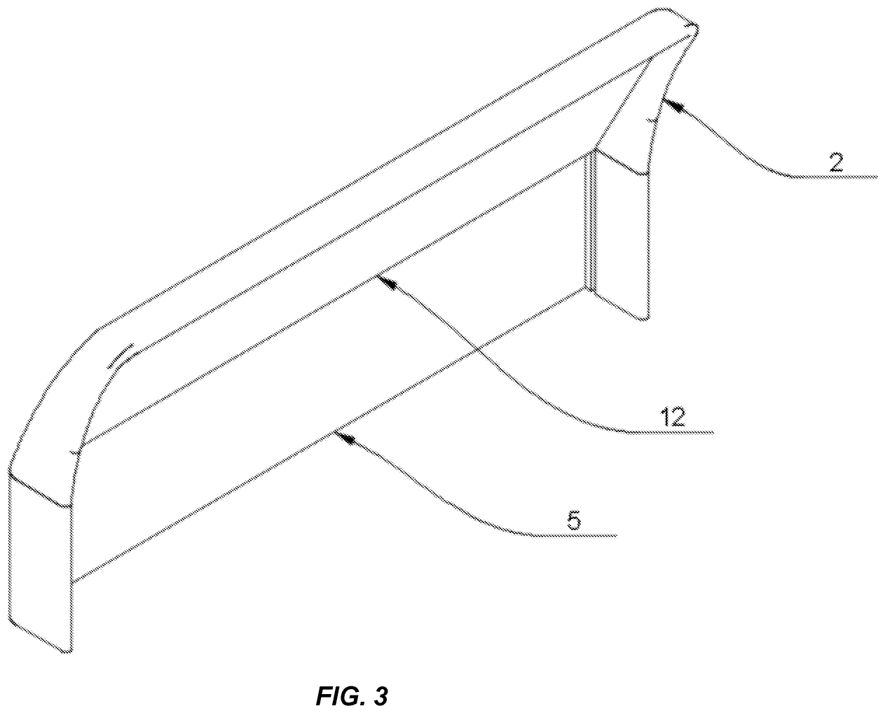

As shown in FIGS. 1 and 3, some embodiments may incorporate an optional reflector 2 that may be placed opposite the common touch point 3 from the enclosure 1 to ensure sufficient illumination using a UVC-reflective inner surface 12. Reflector 2 may comprise plastic or other lightweight, durable material. Embodiments of the reflector 2 may also include an optional extender 5 attached to the base of the reflector 2 for improved coverage by the UVC light. As with the enclosure 1, the base of the reflector 2 or extender 5 may be affixed to the surface adjacent to the common touchpoint 3 opposite to and facing the enclosure 1 in any suitable manner and configuration.

For embodiments where an existing, convenient power source is accessible, such as, when the automatic sanitizer 20 is attached to an ATM or gas pump, power source 6 may comprise a wired circuit or other plug into the existing power source in lieu of batteries, in such embodiments, the low-battery indicator 7 may be omitted, or may be replaced with a powered-on indicator or the like.

As will be apparent to those of ordinary skill in the art having the benefit of this disclosure, the advantages of the automatic sanitizer 20 include, but are not limited to, lower cost, lighter weight, easier installation, and longer life of the UV source than other current touch point sanitization methods. Likewise, the disclosed automatic sanitizer 20 is a motion-activated UV light for sanitizing common touch points 3 with intrinsic safety methods to prevent excessive UV exposure to humans.

Although various embodiments have been shown and described, the present disclosure is not so limited and will be understood to include all such modifications and variations as would be apparent to one skilled in the art.

What is claimed is:

1. An automatic sanitizer for use near a common human touch point, the automatic sanitizer comprising:
a power source to provide power to operate the automatic sanitizer;
a motion sensor to detect the presence of a human appendage within a field of detection of the motion sensor and to generate a corresponding motion signal; and
a microcontroller circuit that operates at least one LI VC LED to illuminate after a first predetermined amount of time from receipt of the corresponding motion signal and to cease illumination of the at least one UVC LED after a second predetermined amount of time or if presence of a human appendage is detected.

2. The automatic sanitizer of claim 1 further comprising:
an enclosure to house the power source and the microcontroller circuit, at least a portion of the motion sensor, and at least a portion of the at least one UVC LED.

3. The automatic sanitizer of claim 2 further comprising:
an extender mount attached to a base of the enclosure and configured to provide an angle of incidence to illumination from the at least one UVC LED onto the common human touch point near the automatic sanitizer.

4. The automatic sanitizer of claim 1 wherein the power source comprises a battery.

5. The automatic sanitizer of claim 4 further comprising:
a low battery indicator to provide an indication when the battery power will be inadequate to operate.

6. The automatic sanitizer of claim 5 wherein the low battery indicator comprises a light.

7. The automatic sanitizer of claim 2 further comprising:
a safety mechanism to shield human eyes from direct exposure to the illumination from the at least one UVC LED.

8. The automatic sanitizer of claim 7 wherein the safety mechanism comprises an appropriately shaped lip on the enclosure.

9. The automatic sanitizer of claim 1 further comprising:
a reflector positionable near the common human touch point and configured to reflect illumination from the at least one UVC LED onto the common human touch point.

10. An automatic sanitizer system for use near a common human touch point, the automatic sanitizer system comprising:
a power source to provide power to operate an automatic sanitizer device;
a motion sensor on the automatic sanitizer device to detect the presence of a human appendage within a field of detection of the motion sensor and to generate a corresponding motion signal;
a microcontroller circuit in the automatic sanitizer device that operates at least one UVC LED to illuminate after a first predetermined amount of time from receipt of the corresponding motion signal and to cease illumination of the at least one UVC LED after a second predetermined amount of time or if presence of a human appendage is detected; and
a reflector positionable near the common human touch point across from the automatic sanitizer device and configured to reflect illumination from the at least one UVC LED onto the common human touch point.

11. The automatic sanitizer system of claim 10 further comprising:
an extender mount attached to a base of the automatic sanitizer device and configured to provide an angle of incidence to illumination from the at least one UVC LED onto the common human touch point near the automatic sanitizer.

12. The automatic sanitizer system of claim 10 wherein the power source comprises a battery.

13. The automatic sanitizer system of claim 12 further comprising:
a low battery indicator to provide an indication when the battery power will be inadequate to operate.

14. The automatic sanitizer system of claim 13 wherein the low battery indicator comprises a light.

15. The automatic sanitizer system of claim 10 further comprising:
a safety mechanism to shield human eyes from direct exposure to the illumination from the at least one LED.

16. The automatic sanitizer of claim 15 wherein the safety mechanism comprises an appropriately shaped lip on an enclosure of the automatic sanitizer device.
